(19) United States
(12) Patent Application Publication
(10) Pub. No.: US 2024/0277289 A1
(43) Pub. Date: Aug. 22, 2024

(54) METHOD FOR OUTPUTTING MESSAGE SUGGESTING BEHAVIOR TO USER ON BASIS OF BLOOD GLUCOSE VALUES OF USER, AND ELECTRONIC DEVICE PERFORMING SAME

(71) Applicant: SAMSUNG ELECTRONICS CO., LTD., Suwon-si (KR)

(72) Inventors: Seongho CHO, Suwon-si (KR); Hyogil KIM, Suwon-si (KR); Minhee HWANG, Suwon-si (KR)

(21) Appl. No.: 18/653,525

(22) Filed: May 2, 2024

Related U.S. Application Data
(63) Continuation of application No. PCT/KR2022/012691, filed on Aug. 25, 2022.

(30) Foreign Application Priority Data
Nov. 2, 2021 (KR) 10-2021-0148988
Nov. 30, 2021 (KR) 10-2021-0168005

(51) Int. Cl. A61B 5/00; A61B 5/0205; A61B 5/11; A61B 5/145

(52) U.S. Cl. CPC A61B 5/486; A61B 5/02055; A61B 5/14532; A61B 5/681; A61B 5/7282; A61B 5/7285; A61B 5/1123; A61B 5/4866

(57) ABSTRACT

An electronic device may include a blood glucose sensor configured to generate a blood glucose value by measuring a blood glucose level of a user, at least one processor, and memory storing instructions that, when executed by the at least one processor individually and/or collectively, cause the electronic device to generate logs for the blood glucose value of the user obtained through the blood glucose sensor, determine a target event based on a target blood glucose value of the user obtained through the blood glucose sensor and the logs, and output a message suggesting a target behavior for the target event.

### CROSS-REFERENCE TO RELATED APPLICATIONS

This application is a continuation application of International Application No. PCT/KR2022/012691 designating the United States, filed on Aug. 25, 2022, in the Korean Intellectual Property Receiving Office and claiming priority to Korean Patent Application No. 10-2021-0148988, filed on Nov. 2, 2021 and Korean Patent Application No. 10-2021-0168005, filed on Nov. 30, 2021, in the Korean Intellectual Property Office, the disclosures of which are all hereby incorporated by reference herein in their entireties.

### BACKGROUND

### 1. Field

Various example embodiments relate to technology for outputting a message suggesting a behavior to a user.

### 2. Description of Related Art

Continuous glucose monitoring systems use blood glucose measurement methods using an electrochemical scheme that requires invasive withdrawal of blood and/or blood glucose measurement methods using a minimally invasive body-attached glucose meter. The invasive methods periodically measure blood glucose levels through an electrochemical scheme with a user doing a periodical finger prick to collect whole blood, or continuously measure blood glucose levels in interstitial fluid using a biosensor implanted under the skin. The minimally invasive methods may continuously measure blood glucose values by sensing glucose oxidase using a needle inserted under the skin. In addition, techniques for continuously measuring the blood glucose level of a user in a wearable device such as a watch or a contact lens have been developed or are being reviewed. As non-invasive blood glucose measurement methods, techniques such as a method of measuring the amount of glucose in the blood using Raman spectroscopy on the waves reflected by irradiating a laser to capillaries, a method of measuring the blood glucose level based on the reflected energy spectrum generated by applying energy to glucose in the blood using optical and/or ultrasonic technology, and a method of estimating the blood glucose level based on the amount of glucose in body fluid are also being developed.

With the spread of such techniques for measuring the blood glucose level of a user, it became relatively easy to measure blood glucose levels continuously, and there have been a growing number of methods that allow diabetic patients or prediabetic users who need to manage their blood glucose levels continuously and even normal users to measure and manage their blood glucose levels that change daily.

### SUMMARY

Invasive blood glucose measurement methods and minimally invasive blood glucose measurement methods have measurement errors in blood glucose values due to hemic differences between arterial blood, capillary blood, and/or venous blood, and depending on the blood glucose measurement sensor, measurement errors may occur up to about 20 to 30%. Additionally, the invasive blood glucose measurement methods may have errors in the measured blood glucose values when a user is not accurately aware of the correct method of measuring a blood glucose level. Services provided based on absolute blood glucose values and services using relative changes in blood glucose values measured by the same sensor may help provide useful information to users. The invasive or minimally invasive blood glucose measurement methods are used by patients who want to actively manage their blood glucose levels, but with the development of non-invasive blood glucose measurement methods, people other than diabetic patients may also measure changes in blood glucose levels in their daily lives. At this time, due to the technical limitations of non-invasive blood glucose measurement methods, a method of obtaining accurate blood glucose values and a method of tracking relative changes in blood glucose levels may be used. In this environment, by continuously measuring blood glucose values of users or changes in relative blood glucose values, useful services related to that may be provided to the users.

An example embodiment may provide an electronic device for outputting a message suggesting a behavior to a user based on the blood glucose value of the user.

However, the technical goals are not limited to those described above, and other technical goals may be present.

According to various example embodiments, an electronic device may include a blood glucose sensor configured to generate a blood glucose value at least by measuring a blood glucose level of a user, and at least one processor configured to control the electronic device, wherein the at least one processor may be individually and/or collectively configured to generate logs for the blood glucose value of the user obtained through the blood glucose sensor, determine a target event based on a target blood glucose value of the user obtained through the blood glucose sensor and the logs, and control to output a message suggesting a target behavior for the target event.

According to various example embodiments, a method, performed by an electronic device, may include generating logs for a blood glucose value of a user obtained through a blood glucose sensor of the electronic device, determining a target event based on a target blood glucose value of the user obtained through the blood glucose sensor and the logs, and outputting a message suggesting a target behavior for the target event.

### EFFECTS

According to various example embodiments, an electronic device for outputting a message suggesting a behavior to a user based on a blood glucose value of the user.

### BRIEF DESCRIPTION OF DRAWINGS

The above and other aspects, features, and advantages of certain example embodiments will be more apparent from the following detailed description, taken in conjunction with the accompanying drawings, in which:

FIG. 1 is a block diagram of an electronic device in a network environment according to various example embodiments.

FIGS. 2A and 2B are perspective views of an electronic device according to an example embodiment.

FIG. 3 is an exploded perspective view of an electronic device according to an example embodiment.

FIG. 4 is a flowchart of a method of outputting a message suggesting a behavior to a user according to an example embodiment.

FIG. 5 illustrates changes in blood glucose values of normal people and diabetic patients according to an example.

FIG. 6 is a flowchart of a method of generating logs for a blood glucose value of a user according to an example embodiment.

FIG. 7 illustrates a method of generating a log based on a type of a behavior according to an example.

FIG. 8 is a flowchart of a method of setting an average blood glucose range for a user according to an example embodiment.

FIG. 9 is a flowchart of a method of generating a log for a behavior based on biometric information of a user according to an example embodiment.

FIG. 10 illustrates logs for a behavior including biometric information according to an example.

FIG. 11 is a flowchart of a method of determining a target event to be having an abnormal meal based on the highest blood glucose value according to an example embodiment.

FIG. 12 is a flowchart of a method of determining a target event to be having an abnormal meal based on a blood glucose reduction time according to an example embodiment.

FIG. 13 is a flowchart of a method of determining a target event to be excessively exercising according to an example embodiment.

FIG. 14 is a flowchart of a method of outputting a message to a user according to an example embodiment.

FIG. 15 illustrates messages output to a user according to an example embodiment.

FIG. 16 is a flowchart of a method of outputting a suggestion message when a preset condition is satisfied according to an example embodiment.

FIG. 17 is a configuration diagram of a system for outputting a message suggesting a behavior to a user according to an example embodiment.

### DETAILED DESCRIPTION

Hereinafter, various embodiments of the present disclosure will be described with reference to the accompanying drawings. However, this is not intended to limit the present disclosure to specific embodiments, and it should be understood that various modifications, equivalents, and/or alternatives of the embodiments of the present disclosure are included.

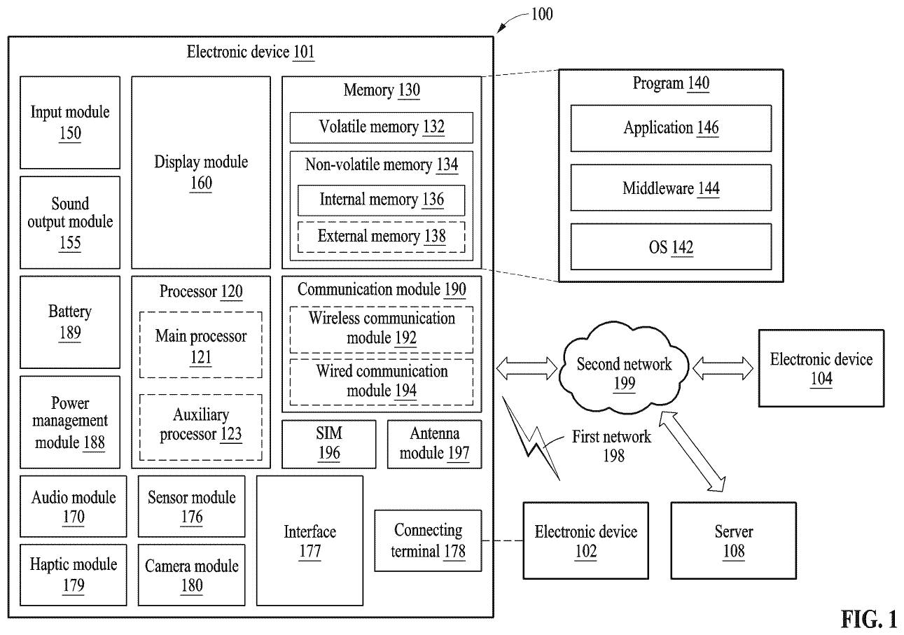

FIG. 1 is a block diagram of an electronic device in a network environment according to various embodiments.

FIG. 1 is a block diagram illustrating an electronic device 101 in a network environment 100 according to various embodiments. Referring to FIG. 1, the electronic device 101 in the network environment 100 may communicate with an electronic device 102 via a first network 198 (e.g., a short-range wireless communication network), or communicate with at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one (e.g., the connecting terminal 178) of the above components may be omitted from the electronic device 101, or one or more other components may be added in the electronic device 101. In some embodiments, some (e.g., the sensor module 176, the camera module 180, or the antenna module 197) of the components may be integrated as a single component (e.g., the display module 160).

The processor 120 may execute, for example, software (e.g., a program 140) to control at least one other component (e.g., a hardware or software component) of the electronic device 101 connected, directly or indirectly, to the processor 120, and may perform various data processing or computation. According to an embodiment, as at least a portion of data processing or computation, the processor 120 may store a command or data received from another component (e.g., the sensor module 176 or the communication module 190) in a volatile memory 132, process the command or the data stored in the volatile memory 132, and store resulting data in a non-volatile memory 134. According to an embodiment, the processor 120 may include a main processor 121 (e.g., a central processing unit (CPU) or an application processor (AP)) or an auxiliary processor 123 (e.g., a graphics processing unit (GPU), a neural processing unit (NPU), an image signal processor (ISP), a sensor hub processor, or a communication processor (CP)) that is operable independently of, or in conjunction with the main processor 121. For example, when the electronic device 101 includes the main processor 121 and the auxiliary processor 123, the auxiliary processor 123 may be adapted to consume less power than the main processor 121 or to be specific to a specified function. The auxiliary processor 123 may be implemented as separate from, or as part of the main processor 121.

The auxiliary processor 123 may control at least some of functions or states related to at least one (e.g., the display module 160, the sensor module 176, or the communication module 190) of the components of the electronic device 101, instead of the main processor 121 while the main processor 121 is in an inactive (e.g., sleep) state, or together with the main processor 121 while the main processor 121 is an active state (e.g., executing an application). According to an embodiment, the auxiliary processor 123 (e.g., an ISP or a CP) may be implemented as a portion of another component (e.g., the camera module 180 or the communication module 190) that is functionally related to the auxiliary processor 123. According to an embodiment, the auxiliary processor 123 (e.g., an NPU) may include a hardware structure specified for artificial intelligence (AI) model processing. An artificial intelligence model may be generated through machine learning. Such learning may be performed, e.g., by the electronic device 101 where the artificial intelligence is performed, or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network, or a combination of two or more thereof, but is not limited thereto. The artificial intelligence model may, additionally or alternatively, include a software structure other than the hardware structure.

Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The memory 130 may store various pieces of data used by at least one component (e.g., the processor 120 or the sensor module 176) of the electronic device 101. The various pieces of data may include, for example, software (e.g., the program 140) and input data or output data for a command related thereto. The memory 130 may include the volatile memory 132 or the non-volatile memory 134.

The program 140 may be stored as software in the memory 130, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing a record. The receiver may be used to receive an incoming call. According to an embodiment, the receiver may be implemented separately from the speaker or as a portion of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to sense a touch, or a pressure sensor adapted to measure an intensity of a force incurred by the touch.

The audio module 170 may convert a sound into an electrical signal or vice versa. According to an embodiment, the audio module 170 may obtain the sound via the input module 150 or output the sound via the sound output module 155 or an external electronic device (e.g., an electronic device 102 such as a speaker or a headphone) directly or wirelessly connected to the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, an illuminance sensor, or a blood glucose sensor.

According to an embodiment, the blood glucose sensor may measure the blood glucose value of the user or changes in the blood glucose value using a non-invasive glucose monitoring (NGM) method.

For example, the blood glucose sensor may irradiate a laser to the skin of the user through Raman spectroscopy and measure (or sense) the blood glucose value based on the unique vibration of the laser changed by reflection. As another example, the blood glucose sensor may sense the blood glucose value using optics and ultrasound. When the blood glucose sensor irradiates light from an infrared light source to the skin, the blood glucose sensor may measure the blood glucose value by sensing ultrasonic waves generated as blood glucose molecules in the blood absorb the light. As still another example, the blood glucose sensor may measure the blood glucose value by irradiating near-infrared light to the veins of the user and analyzing the reflected light. The methods of non-invasively measuring the blood glucose value of the user used by the blood glucose sensor are not limited to the described embodiments.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high-definition multimedia interface (HDMI), a universal serial bus (USB) interface, a secure digital (SD) card interface, or an audio interface.

The connecting terminal 178 may include a connector via which the electronic device 101 may be physically connected, directly or indirectly, to an external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or an electrical stimulus which may be recognized by a user via his or her tactile sensation or kinesthetic sensation. According to an embodiment, the haptic module 179 may include, for example, a motor, a piezoelectric element, or an electric stimulator.

The camera module 180 may capture a still image and moving images. According to an embodiment, the camera module 180 may include one or more lenses, image sensors, ISPs, or flashes.

The power management module 188 may manage power supplied to the electronic device 101. According to an embodiment, the power management module 188 may be implemented as, for example, at least a portion of a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that operate independently of the processor 120 (e.g., an application processor) and support direct (e.g., wired) communication or wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module, or a power line communication (PLC) module). A corresponding one of these communication modules may communicate with the external electronic device 104 via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., a LAN or a wide area network (WAN))). These various types of communication modules may be implemented as a single component (e.g., a single chip), or may be implemented as multiple components (e.g., multiple chips) separate from each other. The wireless communication module 192 may identify and authenticate the electronic device 101 in a communication network, such as the first network 198 or the second network 199, using subscriber information (e.g., international mobile subscriber identity (IMSI)) stored in the SIM 196.

The wireless communication module 192 may support a 5G network after a 4G network, and a next-generation communication technology, e.g., a new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., a mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), an array antenna, analog beamforming, or a large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., an external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in a communication network, such as the first network 198 or the second network 199, may be selected by, for example, the communication module 190 from the plurality of antennas. The signal or the power may be transmitted or received between the communication module 190 and the external electronic device via the at least one selected antenna. According to some embodiments, another component (e.g., a radio frequency integrated circuit (RFIC)) other than the radiating element may be additionally formed as a portion of the antenna module 197.

According to various embodiments, the antenna module 197 may form a mm Wave antenna module. According to an embodiment, the mm Wave antenna module may include a PCB, an RFIC disposed on a first surface (e.g., a bottom surface) of the PCB or adjacent to the first surface and capable of supporting a designated a high-frequency band (e.g., the mm Wave band), and a plurality of antennas (e.g., array antennas) disposed on a second surface (e.g., a top or a side surface) of the PCB, or adjacent to the second surface and capable of transmitting or receiving signals in the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the external electronic devices 102 and 104 may be a device of the same type as or a different type from the electronic device 101. According to an embodiment, all or some of operations to be executed by the electronic device 101 may be executed at one or more of external electronic devices (e.g., the external electronic devices 102 and 104, or the server 108). For example, if the electronic device 101 needs to perform a function or a service automatically, or in response to a request from a user or another device, the electronic device 101, instead of, or in addition to, executing the function or the service, may request one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an Internet-of-things (IoT) device. The server 108 may be an intelligent server using machine learning and/or a neural network. according to an embodiment, the external electronic device 104 or the server 108 may be included in the second network 199. The electronic device 101 may be applied to intelligent services (e.g., a smart home, a smart city, a smart car, or healthcare) based on 5G communication technology or IoT-related technology.

The electronic device according to embodiments may be one of various types of The electronic device may include, for example, a portable electronic devices. communication device (e.g., a smart phone), a computer device, a portable multimedia device, a portable medical device, a camera, a wearable device, or a home appliance device. According to an embodiment of the disclosure, the electronic device is not limited to those described above.

It should be appreciated that embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “at least one of A, B, or C”, each of which may include any one of the items listed together in the corresponding one of the phrases, or all possible combinations thereof. Terms such as “first”, “second”, or “first” or “second” may simply be used to distinguish the component from other components in question, and do not limit the components in other aspects (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., by wire), wirelessly, or via at least a third element(s) therebetween.

As used in connection with various embodiments of the disclosure, the term “module” may include a unit implemented in hardware, software, or firmware, and may interchangeably be used with other terms, for example, “logic”, “logic block”, “part”, or “circuitry”. A module may be a single integral component, or a minimum unit or part thereof, adapted to perform one or more functions. For example, according to an embodiment, the module may be implemented in a form of an application-specific integrated circuit (ASIC). Thus, each “module” herein may comprise circuitry.

Embodiments as set forth herein may be implemented as software (e.g., the program 140) including one or more instructions that are stored in a storage medium (e.g., an internal memory 136 or an external memory 138) that is readable by a machine (e.g., the electronic device 101). For example, a processor (e.g., the processor 120) of the machine (e.g., the electronic device 101) may invoke at least one of the one or more instructions stored in the storage medium, and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments disclosed herein may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to embodiments, one or more of the above-described components or operations may be omitted, or one or more other components or operations may be added Alternatively or additionally, a plurality of components (e.g., modules or programs) may be integrated into a single component. In such a case, according to embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

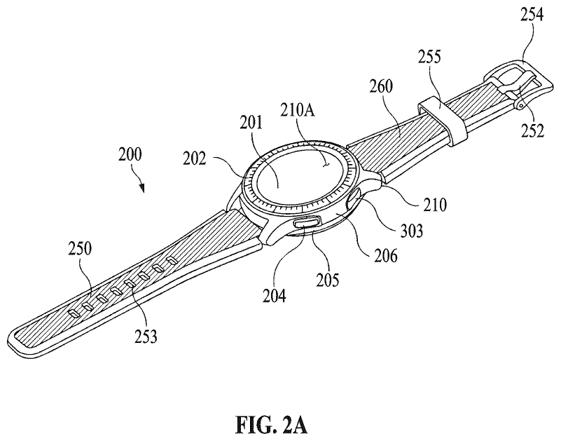

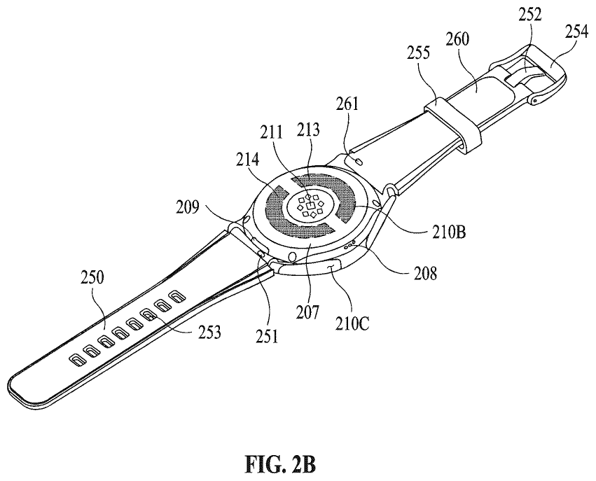

FIGS. 2A and 2B are perspective views of an electronic device according to an embodiment.

Referring to FIGS. 2A and 2B, according to an embodiment, an electronic device 200 (e.g., the electronic device 101 of FIG. 1) may include a housing 210 including a first surface (or a front surface) 210A, a second surface (or a rear surface) 210B, and a side surface 210C surrounding a space between the first surface 210A and the second surface 210B, and fastening members 250 and 260 connected, directly or indirectly, to at least a portion of the housing 210 and configured to detachably attach the electronic device 200 to a body part (e.g., a wrist, or an ankle) of a user. In another embodiment (not shown), the housing may also refer to a structure that forms a portion of the first surface 210A, the second surface 210B, and the side surface 210C of FIG. 2A. According to an embodiment, the first surface 210A may be formed by a front plate 201 (e.g., a glass plate or a polymer plate including various coating layers) of which at least a portion is substantially transparent. The second surface 210B may be formed by a rear plate 207 that is substantially opaque. The rear plate 207 may be formed of, for example, coated or colored glass, ceramic, polymer, metal (e.g., aluminum, stainless steel (SS), or magnesium), or a combination of at least two thereof. The side surface 210C may be coupled, directly or indirectly, to the front plate 201 and the rear plate 207 and may be formed by a side bezel structure (or a “side member”) 206 including a metal and/or a polymer. In some embodiments, the rear plate 207 and the side bezel structure 206 may be integrally formed and may include the same material (e.g., a metal material such as aluminum). The fastening members 250 and 260 may be formed of various materials and may have various shapes. For example, the fastening members 250 and 260 may be formed of woven fabric, leather, rubber, urethane, metal, ceramic, or a combination of at least two of the aforementioned materials and may be implemented in an integrated form or with a plurality of unit links that are movable relative to each other.

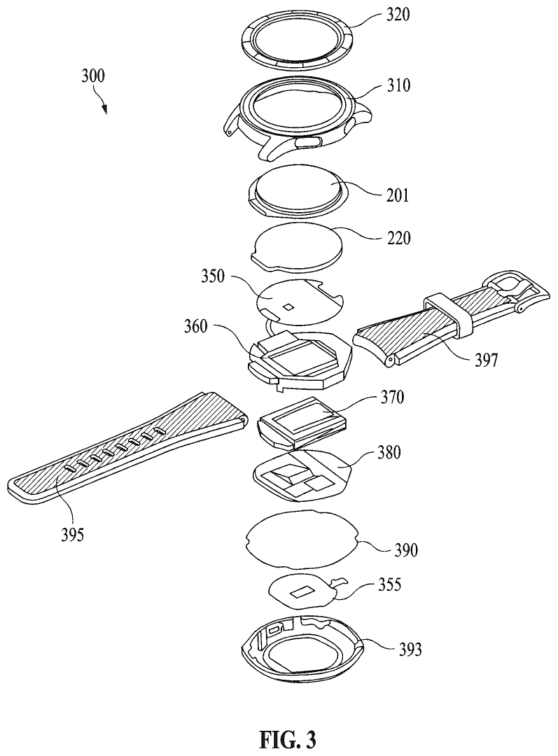

According to an embodiment, the electronic device 200 may include at least one of a display 220 (refer to FIG. 3), audio modules 205 and 208, a sensor module 211, key input devices 202, 203, and 204, a connector hole 209, and a processor (not shown) (e.g., the processor 120 of FIG. 1). In some embodiments, the electronic device 200 may not include at least one (e.g., the key input devices 202, 203, and 204, the connector hole 209, or the sensor module 211) of the components, or additionally include other components.

The display 220 may be exposed through, for example, some portions of the front plate 201. The display 220 may have a shape corresponding to a shape of the front plate 201, and may have various shapes such as a circle, an oval, or a polygon. The display 220 may be coupled to or disposed adjacent to a touch sensing circuit, a pressure sensor capable of measuring an intensity (or pressure) of a touch, and/or a fingerprint sensor.

The audio modules 205 and 208 may include a microphone hole 205 and a speaker hole 208. A microphone for acquiring an external sound may be disposed in the microphone hole 205. In some embodiments, a plurality of microphones may be disposed to detect a direction of a sound. The speaker hole 208 may be used as an external speaker and a call receiver for calls. In some embodiments, the speaker hole 208 and the microphone hole 205 may be implemented as a single hole, or a speaker (e.g., a piezo speaker) may be included without the speaker hole 208.

The sensor module 211 may generate an electrical signal or a data value corresponding to an internal operating state of the electronic device 200 or an external environmental state. The sensor module 211 may include, for example, a biometric sensor module 211 (e.g., a heart rate monitor (HRM) sensor) disposed on the second surface 210B of the housing 210. The electronic device 200 may further include at least one of sensor modules (not shown), for example, a gesture sensor, a gyro sensor, an atmospheric pressure sensor, a magnetic sensor, an acceleration sensor, a grip sensor, a color sensor, an infrared (IR) sensor, a biometric sensor, a temperature sensor, a humidity sensor, or an illuminance sensor.

The sensor module 211 may include electrode areas 213 and 214 that form a portion of the surface of the electronic device 200 and a biosignal detection circuit (not shown) electrically connected, directly or indirectly, to the electrode areas 213 and 214. For example, the electrode areas 213 and 214 may include a first electrode area 213 and a second electrode area 214 disposed on the second surface 210B of the housing 210. The sensor module 211 may be configured such that the electrode areas 213 and 214 obtain an electrical signal from a body part of the user, and the biosignal detection circuit detects biometric information of the user based on the electrical signal.

The key input devices 202, 203, and 204 may include a wheel key 202 disposed on the first surface 210A of the housing 210 and rotatable in at least one direction, and/or side key buttons 203 and 204 disposed on the side surface 210C of the housing 210. The wheel key 202 may have a shape corresponding to the shape of the front plate 202. In another embodiment, the electronic device 200 may not include some or all of the above-described key input devices 202, 203, and 204, and the key input devices 202, 203, and 204 that are not included may be implemented in other forms such as soft keys on the display 220. The connector hole 209 may include another connector hole (not shown) that accommodates a connector (e.g., a universal serial bus (USB) connector) for transmitting and receiving power and/or data to and from an external electronic device and accommodates a connector for transmitting and receiving an audio signal to and from an external electronic device. The electronic device 200 may further include, for example, a connector cover (not shown) that covers at least a portion of the connector hole 209 and blocks infiltration of external foreign materials into the connector hole.

The processor may control the operation of the elements of the electronic device 200.

The fastening members 250 and 260 may be detachably fastened to at least a partial area of the housing 210 using locking members 251 and 261. The fastening members 250 and 260 may include one or more of a fixing member 252, a fixing member fastening hole 253, a band guide member 254, and a band fixing ring 255.

The fixing member 252 may be configured to fix the housing 210 and the fastening members 250 and 260 to a part (e.g., a wrist, an ankle, etc.) of the user's body. The fixing member fastening hole 253 may correspond to the fixing member 252 to fix the housing 210 and the fastening members 250 and 260 to the part of the user's body. The band guide member 254 may be configured to limit a range of a movement of the fixing member 252 when the fixing member 252 is fastened to the fixing member fastening hole 253, so that the fastening members 250 and 260 may be fastened to the part of the user's body in a state of being brought into close contact with the part of the user's body. The band fixing ring 255 may limit a range of a movement of the fastening member 250, 260 in a state in which the fixing member 252 and the fixing member fastening hole 253 are fastened with each other.

FIG. 3 is an exploded perspective view of an electronic device according to an embodiment.

Referring to FIG. 3, an electronic device 300 (e.g., the electronic device 101 of FIG. 1 or the electronic device 200 of FIGS. 2A and 2B) may include a side bezel structure 310, a wheel key 320, a front plate 201, a display 220, a first antenna 350, a second antenna 355, a support member 360 (e.g., a bracket), a battery 370, a PCB 380, a sealing member 390, a rear plate 393, and fastening members 395 and 397. At least one of the components of the electronic device 300 may be the same as or similar to at least one of the components of the electronic device 101 of FIG. 1 or the electronic device 200 of FIGS. 2A and 2B, and a repeated description thereof will be omitted hereinafter. The support member 360 may be disposed inside the electronic device 300 and connected, directly or indirectly, to the side bezel structure 310, or may be integrally formed with the side bezel structure 310. The support member 360 may be formed of, for example, a metal material and/or a non-metal material (e.g., polymer). The display 220 may be connected, directly or indirectly, to one surface of the support member 360, and the PCB 380 may be connected, directly or indirectly, to another surface of the support member 360. The PCB 380 may be provided with a processor, a memory, and/or an interface mounted thereon. The processor may include, for example, one or more of a CPU, a GPU, an AP, a sensor processor, or a communication processor.

The memory may include, for example, a volatile memory or a non-volatile memory. The interface may include, for example, an HDMI, a USB interface, an SD card interface, or an audio interface. For example, the interface may electrically or physically connect the electronic device 300 to an external electronic device, and may include a USB connector, an SD card/multimedia card (MMC) connector, or an audio connector.

The battery 370, which is a device for supplying power to at least one component of the electronic device 300, may include, for example, a non-rechargeable primary battery, a rechargeable secondary battery, or a fuel cell. For example, at least a portion of the battery 370 may be disposed on substantially the same plane as the PCB 380. The battery 370 may be disposed integrally inside the electronic device 200, or disposed detachably from the electronic device 200.

The first antenna 350 may be disposed between the display 220 and the support member 360. The first antenna 350 may include, for example, a near-field communication (NFC) antenna, a wireless charging antenna, and/or a magnetic secure transmission (MST) antenna. For example, the first antenna 350 may perform short-range communication with an external device, wirelessly transmit and receive power used for charging, or transmit a magnetism-based signal including a short-range communication signal or payment data. In another embodiment, an antenna structure may be formed by a portion of the side bezel structure 310 and/or the support member 360, or a combination thereof.

The second antenna 355 may be disposed between the PCB 380 and the rear plate 393. The second antenna 355 may include, for example, an NFC antenna, a wireless charging antenna, and/or an MST antenna. For example, the second antenna 355 may perform short-range communication with an external device, wirelessly transmit and receive power used for charging, or transmit a magnetism-based signal including a short-range communication signal or payment data. In another embodiment, an antenna structure may be formed by a portion of the side bezel structure 310 and/or the rear plate 393, or a combination thereof.

The sealing member 390 may be disposed between the side bezel structure 310 and the rear plate 393. The sealing member 390 may be configured to prevent or reduce chances of moisture and foreign materials from being introduced into a space surrounded by the side bezel structure 310 and the rear plate 393 from the outside.

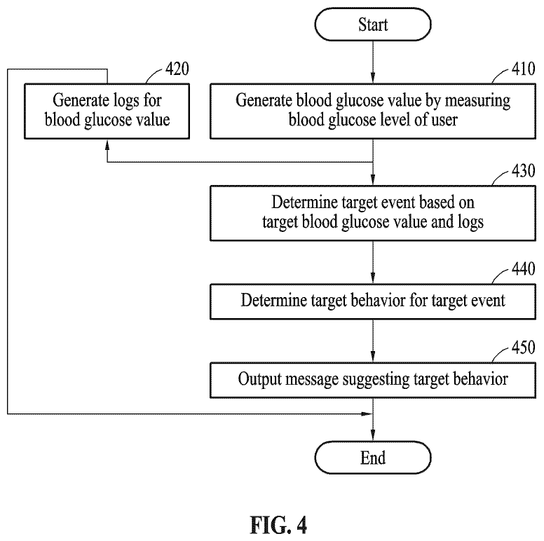

FIG. 4 is a flowchart of a method of outputting a message suggesting a behavior to a user according to an embodiment.

According to an embodiment, a method of outputting a message suggesting a behavior to a user may include operations 410 to 450 below. Operations 410 to 450 may be performed by an electronic device (e.g., the electronic device 101 of FIG. 1, the electronic device 200 of FIGS. 2A and 2B, or the electronic device 300 of FIG. 3).

In operation 410, a blood glucose sensor (e.g., the blood glucose sensor of the sensor module 176 of FIG. 1) of the electronic device may generate a blood glucose value by measuring the blood glucose level of a user. For example, the blood glucose sensor may measure the blood glucose value of the user in a non-invasive or minimally invasive manner. The measured blood glucose value may be an absolute blood glucose level (e.g., in milligrams per deciliter (mg/dL)), or may be a relative value to a preset reference value. For example, the preset reference value may be a fasting blood glucose value recently measured using a blood glucose meter and received from the user, or a blood glucose value received from a blood glucose meter through short-range wireless communication (e.g., Bluetooth Low Energy (BLE)). Even if the measured blood glucose value is a relative value, repeated measurement with the same standard for the same user may show an aspect that the blood glucose level changes, and the blood glucose value measured at a designated time may be used similarly to an absolute blood glucose value. Although blood glucose values are described hereinafter using absolute blood glucose levels, blood glucose values may be relative values to a preset reference value.

According to an embodiment, the blood glucose sensor may operate at a preset operating interval and continuously generate blood glucose values. The operating interval may be adjusted by a processor (e.g., the processor 120 of FIG. 1 and the processor of FIGS. 2A and 2B) of the electronic device. For example, a first blood glucose value may be generated at a first time, and a second blood glucose value may be generated at a second time.

For example, if it is determined that the user is having a meal, the operating interval of the blood glucose sensor may be adjusted so that the blood glucose value is measured at a shorter interval, and if the blood glucose value returns to a normal range (e.g., within 10% of the fasting blood glucose value), the operating interval may be adjusted to the default value.

For example, the electronic device may determine that the user is having a meal when the user inputs the start of a meal into the electronic device. As another example, the electronic device may determine that the user is having a meal using a gesture sensor or gyro sensor in the electronic device. As still another example, the electronic device may receive data (e.g., sound data, image data, and/or sensing data) from a wearable device worn on the face of the user, such as earphones or glasses, and determine that the user is having a meal based on the data.

According to an embodiment, after operation 410 is performed, operations 420 and 430 may be performed independently and in parallel.

In operation 420, the processor of the electronic device may generate logs for the blood glucose value.

According to an embodiment, the processor may determine the type of a behavior performed by the user at the time when the first blood glucose value was measured, and generate a log by associating the time, the type of the behavior, and the first blood glucose value.

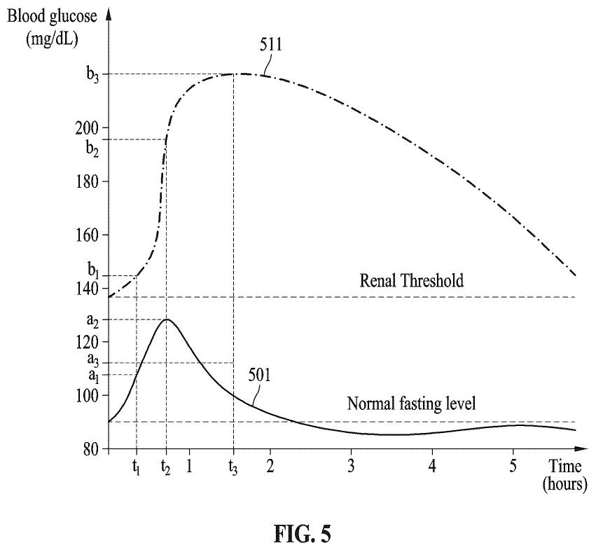

For example, when the blood glucose value increases as the user has a meal, the processor may determine whether the user had a meal based on the blood glucose value and generate a log associated with a meal. The method of generating a log associated with a meal of the user will be described in detail below with reference to FIGS. 5 to 8.

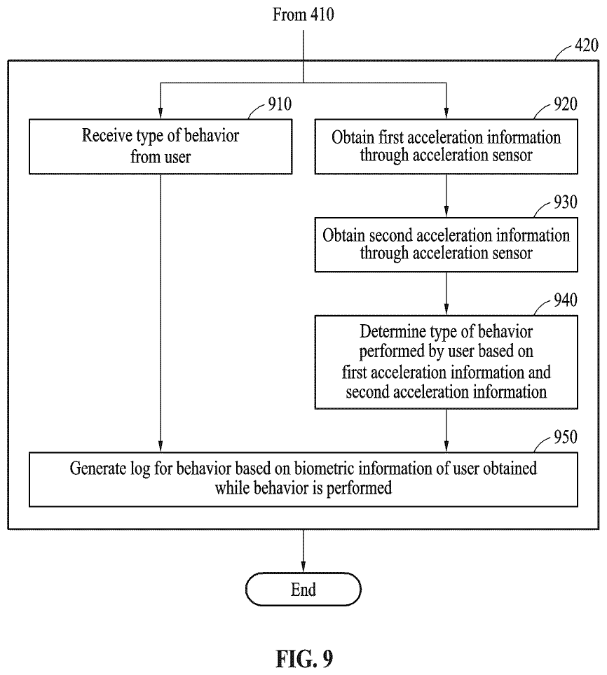

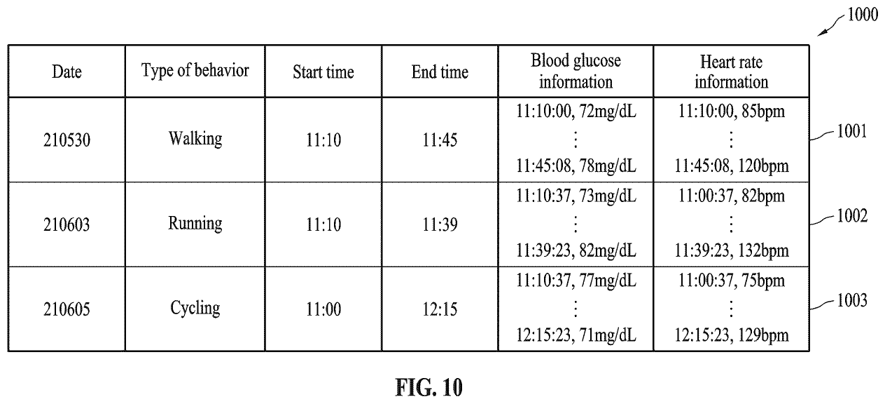

As another example, when the blood glucose value changes as the user exercises, the processor may determine the type of a behavior performed by the user based on at least one of acceleration information measured through an acceleration sensor in the electronic device and biometric information measured through biometric sensors such as a heart rate sensor, pressure sensor, temperature sensor, oxygen saturation sensor, and stress sensor in the electronic device, and generate a log associated with the type of the behavior. The method of generating a log associated with the type of a behavior will be described in detail below with reference to FIGS. 9 and 10.

A blood glucose value may change from time to time depending on the food intake such as a meal or snack of the user. For example, a blood glucose value may begin to rise approximately 10 minutes after starting a meal, reach a peak value about 1 hour after the meal, and return to the value before the meal about 2 to 3 hours after starting the meal.

In addition, a blood glucose value may also change when the user exercises. Since muscles consume glucose during exercise, the liver may generate and supply glucose to the muscles when glucose in the blood is insufficient. For this reason, exercising immediately after a meal may decrease the blood glucose value, whereas exercising on an empty stomach may show an aspect that the blood glucose value decreases and then, slightly increases due to the glucose supplied by the liver. However, exercise increases the amount of glucose consumed by muscles and thus, may have the effect of maintaining a normal blood glucose value at a low level in the long term.

For the above reason, a meal and/or exercise pattern of the user may be estimated using biometric information based on changes in continuously measured blood glucose values. The meal and/or exercise pattern of the user may be stored as logs. The electronic device may determine a lifestyle habit that causes a rapid change in the blood glucose value of the user based on the logs and changes in the blood glucose value, and recommend appropriate behaviors to the user to manage the blood glucose value of the user at an appropriate level.

For example, the processor may generate a message suggesting a behavior to the user and output the message through a display (e.g., the display module 160 of FIG. 1 or the display 220 of FIG. 3). The method of outputting a message to the user based on logs may be performed by operations 430 to 450 described below. Operation 430 may be performed after operation 410 is performed.

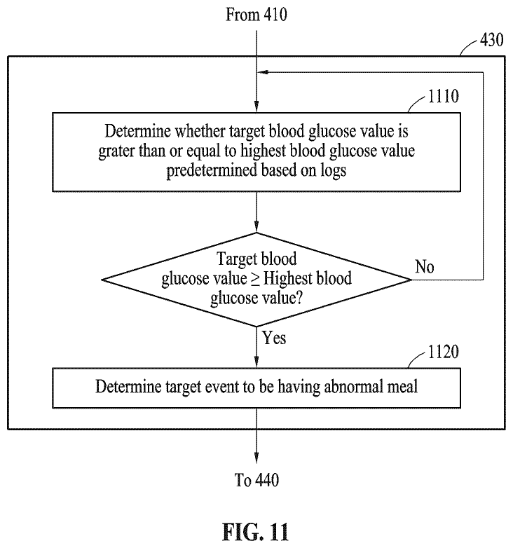

In operation 430, the processor may determine a target event based on a target blood glucose value and logs. The target blood glucose value may be a blood glucose value measured at a designated time (e.g., the most recent time). For example, the target event may include having an abnormal meal and/or excessively exercising. The target event is not limited to the described embodiments. The method of determining a target event based on a target blood glucose value and logs will be described in detail below with reference to FIGS. 11 to 13.

In operation 440, the processor may determine a target behavior for the target event. For example, when the target event is determined to be having an abnormal meal, restricting a designated menu may be determined to be the target behavior. As another example, when the target event is determined to be excessively exercising, reducing the performance time of the exercise may be determined to be the target behavior. The target behavior is not limited to the described embodiments.

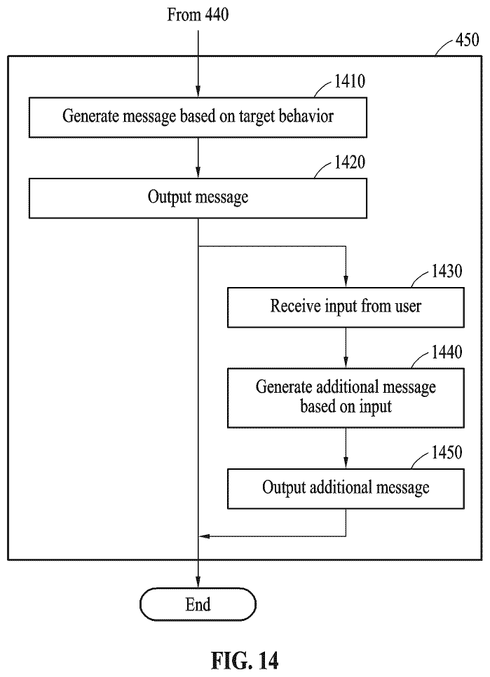

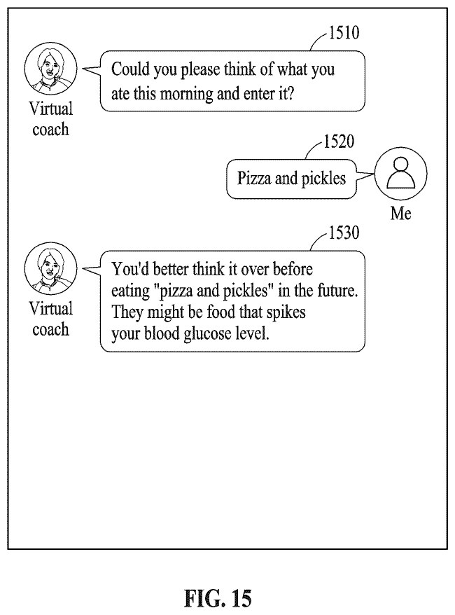

In operation 450, the processor may output a message suggesting the determined target behavior. For example, the processor may generate a message to include one or more of information about the target behavior, information about changes in the blood glucose value, and information about the target event. The method of outputting a message will be described in detail below with reference to FIGS. 14 and 15.

Through the method of outputting a message suggesting a behavior to the user, customized services for improving users' lifestyle habits using continuously measured blood glucose values may be provided to diabetic patients who need to manage blood glucose values and even to normal users.

FIG. 5 illustrates changes in blood glucose values of normal people and diabetic patients according to an example.

In general, diabetic patients may have higher blood glucose values compared to normal people. In particular, a trajectory 511 of blood glucose values of diabetic patients after having a meal may tend to maintain high values for a long time compared to a trajectory 501 of blood glucose values of normal people.

According to an embodiment, the electronic device described above with reference to FIG. 4 (e.g., the electronic device 101 of FIG. 1, the electronic device 200 of FIGS. 2A and 2B, or the electronic device 300 of FIG. 3) may continuously measure blood glucose values of a user. The x-axis of the depicted graph may be a time axis based on the time when the user starts having a meal. For example, it is shown that blood glucose values are measured at times t₁, t₂, and t₃, but the blood glucose values may be measured at intervals of a few seconds or a few minutes.

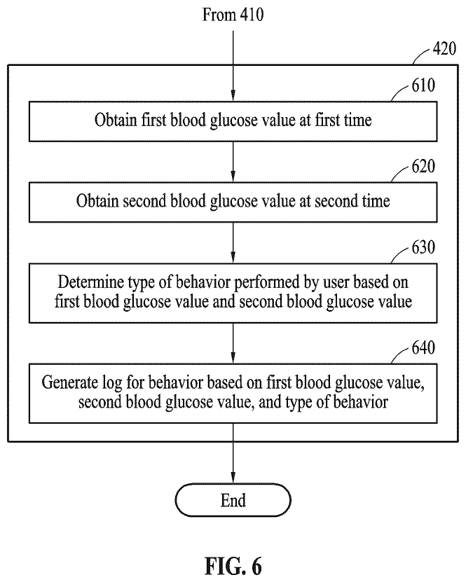

For example, if the user is a normal person, blood glucose values a₁, a₂, and a₃ may be measured at times t₁, t₂, and t₃, respectively. az measured at t₂ may be the peak blood glucose value of the user. As another example, if the user is a diabetic patient, blood glucose values b₁, b₂, and b₃ may be measured at times t₁, t₂, and t₃, respectively. b3 measured at t₃ may be the peak blood glucose value of the user. For each user, a log associated with the meal may be generated. The method of generating a log associated with a meal will be described in detail below with reference to FIGS. 6 to 8.

FIG. 6 is a flowchart of a method of generating logs for a blood glucose value of a user according to an example.

According to an embodiment, operation 420 described above with reference to FIG. 4 may include operations 610 to 640 to be described below. Operations 610 to 640 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 610, the processor may obtain a first blood glucose value at a first time. For example, the processor may receive the first blood glucose value from a blood glucose sensor (e.g., the blood glucose sensor of the sensor module 176 of FIG. 1) of the electronic device.

According to an embodiment, the processor may adjust an operating interval of the blood glucose sensor when the first blood glucose value is above a preset average blood glucose range (e.g., within 10%of the fasting blood glucose value).

In operation 620, the processor may obtain a second blood glucose value at a second time. For example, the processor may receive the second blood glucose value from the blood glucose sensor of the electronic device. For example, the difference between the first time and the second time may be the operating interval of the blood glucose sensor, and is not limited to the described embodiment. Operations 610 and 620 may be operations of continuously monitoring the blood glucose values of the user.

In operation 630, the processor may determine the type of a behavior performed by the user based on the first blood glucose value and the second blood glucose value. The type of the behavior may be determined based on a change pattern of the continuously measured blood glucose values. For example, when the blood glucose value shows a change pattern of remaining within a predetermined range, rising after a predetermined time, and then decreasing, the type of the behavior performed by the user may be determined to be having a meal. As another example, if the blood glucose value shows a change pattern of decreasing and then increasing, the type of the behavior performed by the user may be determined to be exercising.

According to an embodiment, the processor may determine the type of the behavior performed by the user based further on motion information (e.g., acceleration information and/or rotation direction information) about the motion of the electronic device generated by a motion sensor, including an acceleration sensor and/or a gyro sensor.

According to an embodiment, the processor may determine the type of the behavior performed by the user based further on information about the body temperature of the user generated by a body temperature sensor.

In operation 640, the processor may generate a log for the behavior based on the first blood glucose value, the second blood glucose value, and the determined type of the behavior. For example, if the type of the behavior is having breakfast, a log indicating a change in the blood glucose value for breakfast may be generated. As another example, if the type of the behavior is walking, a log indicating a change in the blood glucose value for walking may be generated.

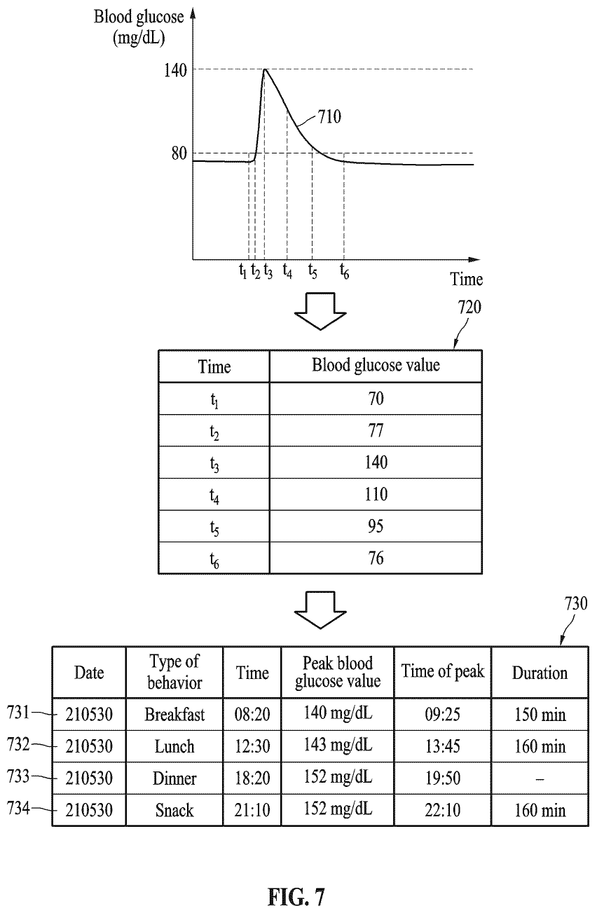

FIG. 7 illustrates a method of generating a log based on a type of a behavior according to an example.

According to an example, a change trajectory 710 of the blood glucose value of a user is shown.

An electronic device (e.g., the electronic device 101 of FIG. 1, the electronic device 200 of FIGS. 2A and 2B, or the electronic device 300 of FIG. 3) may measure blood glucose values 720 of the user at a plurality of times t₁, t₂, t₃, t₄, t₅, and t₆, respectively. Although the blood glucose values 720 are expressed as absolute blood glucose levels (e.g., in mg/dL), the blood glucose values 720 may be expressed as relative values depending on the embodiment.

According to an embodiment, the electronic device may determine the type of a behavior performed by the user to be having a meal based on the blood glucose values 720. For example, the electronic device may determine whether the meal is breakfast, lunch, dinner, or a snack based on the plurality of times t₁, t₂, t₃, t₄, t₅, and t₆.

For example, the processor may determine a start time of the meal based on the time when the measured blood glucose value exceeds an average blood glucose range (e.g., 63 to 77 mg/dL) set based on the fasting blood glucose value, if the fasting blood glucose value of the user is about 70 mg/dL. Subsequently, the processor may determine the time when the blood glucose value reaches the peak value (e.g., 140 mg/dL) to be a peak time, and determine the time when the blood glucose value decreases back to the normal blood glucose value (e.g., the fasting blood glucose value or the average blood glucose range). The period from the start time of the meal to the time when the blood glucose value decreases back to the normal blood glucose value may be determined to be the duration. A plurality of logs 730 may be generated through continuous measurement of blood glucose values. For example, the plurality of logs 730 may include a log 731 for breakfast, a log 732 for lunch, a log 733 for dinner, and/or a log 734 for a snack.

According to an embodiment, the electronic device may receive menu information about the corresponding meals from the user and associate the menu information with the logs.

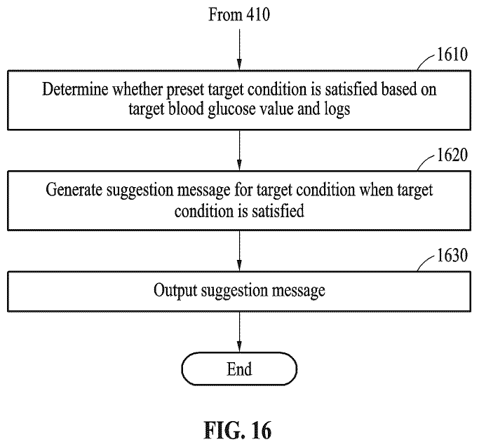

According to an embodiment, the electronic device may evaluate the daily meals of the user based on the plurality of logs 730. An example of evaluating a meal of a user will be described in detail below with reference to FIG. 16.

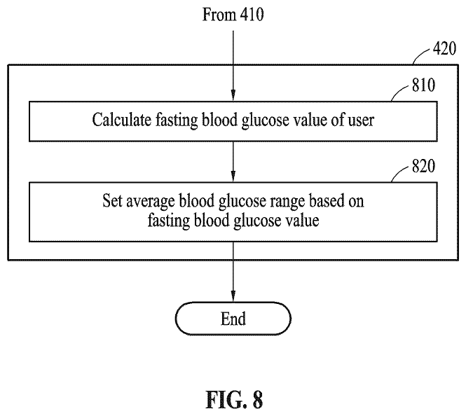

FIG. 8 is a flowchart of a method of setting an average blood glucose range for a user according to an example.

According to an embodiment, operation 420 described above with reference to FIG. 4 may include operations 810 and 820 to be described below. Operations 810 and 820 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3). Operations 810 and 820 may be performed before operations 610 to 640 described with reference to FIG. 6 are performed.

In operation 810, the processor may calculate a fasting blood glucose value for the user. For example, the fasting blood glucose value may be the blood glucose value at a time when the user has not eaten for about 8 hours or more. The processor may determine whether the user is fasting by continuously monitoring the blood glucose value of the user, and calculate the fasting blood glucose value based on the blood glucose value measured in the fasting state.

In operation 820, the processor may set an average blood glucose range based on the fasting blood glucose value. For example, if the fasting blood glucose value of the user is approximately 70 mg/dL, about 63 mg/dL to about 77 mg/dL, which is about 90% to about 110% of the fasting blood glucose value, may be set as the average blood glucose range.

According to an embodiment, the processor may determine whether the user is having a meal or exercising based on the set average blood glucose range and the measured blood glucose value.

FIG. 9 is a flowchart of a method of generating a log for a behavior based on biometric information of a user according to an example.

According to an embodiment, operation 420 described above with reference to FIG. 4 may include operations 910 to 950 to be described below. Operations 910 to 950 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 910, the processor may receive the type of the behavior from the user. For example, the user may input the type of the behavior into the electronic device through a user interface (e.g., a microphone, a button, or a touch sensor) of the electronic device. The received type of the behavior may include at least one of walking, running, cycling, hiking, and swimming, and is not limited to the described embodiments.

According to an embodiment, operations 920 to 940 may be performed regardless of whether operation 910 is performed, or may be preset to be performed when operation 910 is not performed.

In operation 920, the processor may obtain first acceleration information at a third time through an acceleration sensor (e.g., the acceleration sensor of the sensor module 176 of FIG. 1) that measures acceleration information about the motion of the electronic device or the user wearing the electronic device.

In operation 930, the processor may obtain second acceleration information at a fourth time through the acceleration sensor.

In operation 940, the processor may determine the type of the behavior performed by the user based on the first acceleration information and the second acceleration information.

According to an embodiment, the processor may determine the type of the behavior performed by the user based further on information obtained through additional sensors (e.g., a geomagnetic sensor, a gyro sensor, an atmospheric pressure sensor, a grip sensor, a biometric sensor, and/or a temperature sensor) other than the acceleration sensor.

In operation 950, the processor may generate a log for the behavior based on biometric information of the user obtained while the behavior is performed. For example, the biometric information may include at least one of the blood glucose, the heart rate, the body temperature, the blood pressure, the stress level, and the consumed energy of the user.

A log for a behavior related to exercise will be described in detail below with reference to FIG. 10.

FIG. 10 illustrates logs for a behavior including biometric information according to an example.

According to an embodiment, a plurality of logs 1000 may include a first log 1001 for a first behavior, a second log 1002 for a second behavior, and a third log 1003 for a third behavior.

According to an embodiment, an electronic device (e.g., the electronic device 101 of FIG. 1, the electronic device 200 of FIGS. 2A and 2B, or the electronic device 300 of FIG. 3) may determine a type of a behavior (e.g., walking, running, or cycling) based on an input received from a user or obtained acceleration information. Additionally, the electronic device may determine the start time and end time of the behavior.

According to an embodiment, the electronic device may generate the log 1001, 1002, or 1003 for the behavior to further include blood glucose values and biometric information (e.g., the heart rate) obtained during the time while the behavior is performed. Although the heart rate is shown as biometric information, the body temperature, the blood pressure, the stress level, and the consumed energy may be further included in the log.

According to an embodiment, the electronic device may evaluate the exercise effect of the behavior based on blood glucose information and biometric information. The evaluated exercise effect may be used as basic information to recommend an exercise to the user.

FIG. 11 is a flowchart of a method of determining a target event to be having an abnormal meal based on the highest blood glucose value according to an example.

According to an embodiment, operation 430 described above with reference to FIG. 4 may include operations 1110 and 1120 to be described below. Operations 1110 and 1120 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 1110, the processor may determine whether the target blood glucose value is greater than or equal to a predetermined highest blood glucose value based on stored logs. For example, the highest blood glucose value may be determined based on the peak blood glucose values in one or more logs for meals. For example, the maximum or a high value among the peak blood glucose values may be determined to be the highest blood glucose value, or the average of the peak blood glucose values may be determined to be the highest blood glucose value.

In operation 1120, the processor may determine the target event to be having an abnormal meal when the target blood glucose value is greater than or equal to the highest blood glucose value. For example, if the food the user ate is high in sugar content, the measured blood glucose value may exceed the existing highest blood glucose value. To improve the eating habits of the user, the processor may generate an event when the target blood glucose value is greater than or equal to the highest blood glucose value.

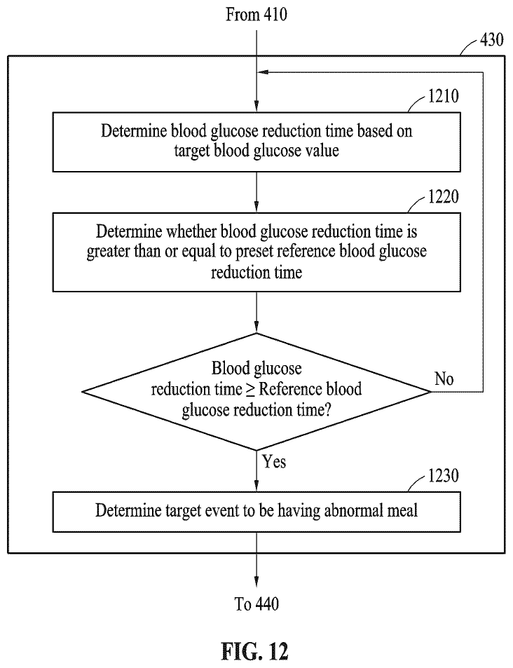

FIG. 12 is a flowchart of a method of determining a target event to be having an abnormal meal based on a blood glucose reduction time according to an example.

According to an embodiment, operation 430 described above with reference to FIG. 4 may include operations 1210 to 1230 to be described below. Operations 1210 to 1230 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 1210, the processor may determine a blood glucose reduction time based on the target blood glucose value. The blood glucose reduction time may be the duration which is the period from the start time of a meal to the time when the blood glucose value decreases back to the normal blood glucose value.

In operation 1220, the processor may determine whether the blood glucose reduction time is greater than or equal to a preset reference blood glucose reduction time. For example, the reference blood glucose reduction time may be determined based on the durations of one or more logs for meals. For example, the average of the durations may be determined to be the reference blood glucose reduction time.

In operation 1230, when the blood glucose reduction time is greater than or equal to the reference blood glucose reduction time, the processor may determine the target event to be having an abnormal meal. For example, if the food the user ate is high in sugar content, the measured blood glucose reduction time may be longer than usual. To improve the eating habits of the user, the processor may generate an event when the blood glucose reduction time is greater than or equal to the reference blood glucose reduction time.

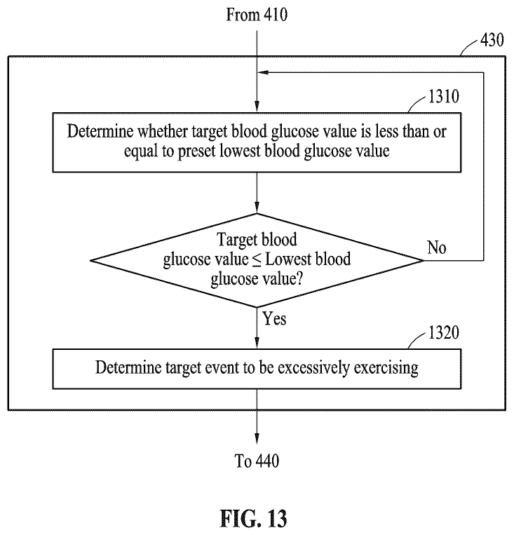

FIG. 13 is a flowchart of a method of determining a target event to be excessively exercising according to an example.

According to an embodiment, operation 430 described above with reference to FIG. 4 may include operations 1310 and 1320 to be described below. Operations 1310 and 1320 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 1310, the processor may determine whether the target blood glucose value is less than or equal to a preset lowest blood glucose value. For example, the lowest blood glucose value may be determined based on blood glucose values in one or more logs for exercise.

In operation 1320, the processor may determine the target event to be excessively exercising when the target blood glucose value is less than or equal to the lowest blood glucose value. For example, when the user excessively exercises, the measured blood glucose value may fall below an appropriate level. To improve the exercising habits of the user, the processor may generate an event when the target blood glucose value is less than or equal to the lowest blood glucose value.

FIG. 14 is a flowchart of a method of outputting a message to a user according to an example.

According to an embodiment, operation 450 described above with reference to FIG. 4 may include operations 1410 to 1450 to be described below. Operations 1410 to 1450 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3).

In operation 1410, the processor may generate a message based on the target behavior.

According to an embodiment, when the event is determined to be having an abnormal meal, the processor may determine restricting the meal or the menu of the meal to be the target behavior, and generate a message suggesting restricting the menu of the meal to the user. For example, when the user has a snack, a message suggesting reducing snacks to the user may be generated.

According to an embodiment, when the event is determined to be excessively exercising, the processor may determine reducing the performance time of the exercise to be the target behavior, and generate a message suggesting reducing the performance time of the exercise to the user.

In operation 1420, the processor may output the message through a display (e.g., the display module 160 of FIG. 1 or the display 220 of FIG. 3) and/or a speaker (e.g., the sound output module 155 of FIG. 1) of the electronic device. The user may recognize undesirable behaviors in his or her lifestyle habits, such as eating habits or exercising habits, through the output message.

According to an embodiment, operations 1430 to 1450 may be further performed as needed. For example, operations 1430 to 1450 may be performed so that the electronic device may provide additional information to the user.

In operation 1430, the processor may request a designated input from the user and receive an input from the user in response to the request, in order to provide the user with additional information. For example, the processor may inquire the user about the menu of the meal determined to be an abnormal meal and receive the menu of the meal from the user.

In operation 1440, the processor may generate an additional message based on the input from the user. For example, an additional message suggesting restricting the intake of the received menu may be generated.

In operation 1450, the processor may output the generated additional message through the display and/or the speaker.

FIG. 15 illustrates messages output to a user according to an example.

According to an embodiment, when a user's breakfast is determined to be an abnormal meal, an electronic device (e.g., the electronic device 101 of FIG. 1, the electronic device 200 of FIGS. 2A and 2B, or the electronic device 300 of FIG. 3) may generate and output a first message 1510 to the user to obtain additional information about the breakfast.

The user may input the menu of the breakfast in the form of a message 1520 as a reply to the first message 1510.

The electronic device may generate and output a second message 1530 based on the received menu of the breakfast. Through the second message 1530, the user may improve his or her eating habits.

FIG. 16 is a flowchart of a method of outputting a suggestion message when a preset condition is satisfied according to an example.

According to an embodiment, operations 1610 to 1630 to be described below may be further performed after operation 410 described above with reference to FIG. 4 is performed. Operations 1610 to 1630 may be performed by a processor (e.g., the processor 120 of FIG. 1, or the processor of FIGS. 2A and 2B) of an electronic device (e.g., the electronic device 101 of FIG. 1, the electronic device 200 of FIGS. 2A and 2B, or the electronic device 300 of FIG. 3). Operations 1610 to 1630 may be performed independently of and in parallel with operations 430 to 450 described above with reference to FIG. 4.

In operation 1610, the processor may determine whether a preset target condition is satisfied based on the target blood glucose value and the stored logs. The target condition may refer to any one of one or more preset conditions.

According to an embodiment, the processor may determine that a set condition is satisfied when the peak blood glucose value for a predetermined meal is lower than the usual peak blood glucose value by more than a reference value.

According to an embodiment, the processor may determine that a set condition is satisfied when an indicator calculated based on continuously measured blood glucose values is improved from the previous indicator.

According to an embodiment, the processor may determine that a set condition is satisfied when the peak blood glucose value for lunch in a state in which the user skips breakfast is higher than the usual peak blood glucose value.

According to an embodiment, the processor may determine that a set condition is satisfied when the duration for a predetermined meal is not determined. For example, if the user eats a snack after lunch, the blood glucose value rises again, and thus, the duration for the lunch may not be determined.

According to an embodiment, the processor may determine that a set condition is satisfied when the fasting blood glucose value measured after the user exercises is lower than the previous fasting blood glucose value.

According to an embodiment, the processor may determine that a set condition is satisfied when the measured fasting blood glucose value is higher than the previous fasting blood glucose value.

In operation 1620, the processor may generate a suggestion message for the target condition when the target condition is satisfied.

According to an embodiment, when the peak blood glucose value for a predetermined meal is lower than the usual peak blood glucose value by more than a reference value, the processor may generate a message recommending the meal. For example, the message may be “Today's lunch looks like a good meal for your health. Please keep up good eating habits.”

According to an embodiment, when an indicator calculated based on continuously measured blood glucose values is improved from the previous indicator, the processor may analyze the diets recorded for a preset period and recommend a diet to the user based on the same. The processor may analyze the recommended amounts of nutrients, such as carbohydrates, proteins, fats, and/or minerals, in the recorded diets, and suggest information about nutrients that need improvement or supplementation to the user through a message. Since the recommended intake ratios of carbohydrates, proteins, and/or fats based on calories are about 50 to 60%, about 20%, and about 20% or less, respectively, the processor may suggest adjusting the ratios of nutrients in the recorded diets to the user. For example, when the diet of the user is excessive in carbohydrates, changing carbohydrates from simple sugars to high-fiber and unrefined grains may be suggested to the user through a message. As another example, when the diet of the user is excessive in fat, avoiding eating trans-fat may be suggested to the user through a message. Additionally, avoiding eating foods containing a lot of saturated fatty acids by more than about 7% of total energy may be suggested to the user.

According to an embodiment, when the peak blood glucose value for lunch in a state in which the user skips breakfast is higher than the usual peak blood glucose value, the processor may generate a message suggesting not skipping a meal. For example, the message may be “Skipping breakfast may lead to overeating at lunch. It's better to try not to skip breakfast if possible.”

According to an embodiment, the processor may generate a message suggesting reducing snacking if the duration for a predetermined meal is not determined. For example, the message may be “Did you by any chance eat a snack after lunch yesterday? Frequent snacking may be detrimental to your health. If you feel hungry often, keep some nuts around and eat light.”

According to an embodiment, when the fasting blood glucose value measured after the user exercises is lower than the previous fasting blood glucose value, the processor may generate a message recommending that exercise. For example, the message may be “As you exercised yesterday, you can become healthier. Cycling once every two days as you did during the day yesterday will make you healthier.”

According to an embodiment, when the measured fasting blood glucose value is higher than the previous fasting blood glucose value, the processor may generate a message suggesting exercise to the user based on the stored logs. For example, the message may be “How about doing the indoor cycling that you did last week, for 30 minutes today?”.

In operation 1630, the processor may output the suggestion message through a display (e.g., the display module 160 of FIG. 1 or the display 220 of FIG. 3) and/or a speaker (e.g., the sound output module 155 of FIG. 1) of the electronic device.

Although it is described above that operations 1610 to 1630 are performed by the electronic device, depending on the embodiment, at least some of operations 1610 to 1630 may be performed by an external electronic device (e.g., the server 108 of FIG. 1) connected, directly or indirectly, to the electronic device. For example, operations 1610 and 1620 may be performed by the external electronic device, and operation 1630 may be performed by the electronic device. In the case where the external electronic device performs operations 1610 and 1620, the electronic device may transmit necessary information to the external electronic device, and the external electronic device may transmit a suggestion message to the electronic device.

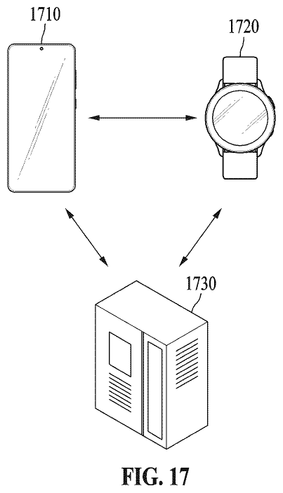

FIG. 17 is a configuration diagram of a system for outputting a message suggesting a behavior to a user according to an example.

According to an embodiment, a system for outputting a message suggesting a behavior to a user may include a first electronic device 1710 (e.g., the electronic device 101 of FIG. 1), a second electronic device 1720 (e.g., the electronic device 200 of FIGS. 2A and FIG. 2B or the electronic device 300 of FIG. 3), and a server 1730 (e.g., the server 108 of FIG. 1). The first electronic device 1710 may be a main device, and the second electronic device 1720 may be a wearable device. The first electronic device 1710 and the second electronic device 1720 may be owned by the same user. For example, a user account of the first electronic device 1710 and a user account of the second electronic device 1720 may be the same.

According to an embodiment, the first electronic device 1710 and the second electronic device 1720 may perform operations 410 to 450 described above with reference to FIG. 4, respectively, or only one electronic device may perform operations 410 to 450.

According to an embodiment, operations 410 to 450 may be performed by a combination of the first electronic device 1710 and the second electronic device 1720. For example, operation 410 may be performed by the second electronic device 1720, and operations 420 to 450 may be performed by the first electronic device 1710. In this case, the second electronic device 1720 may transmit the blood glucose value generated in operation 410 to the first electronic device 1710.

According to an embodiment, the server 1730 may be connected to the first electronic device 1710 and/or the second electronic device 1720, and may perform operations 1610 and 1620 described above with reference to FIG. 16. The first electronic device 1710 or the second electronic device 1720 may output a suggestion message received from the server 1730 to the user.

According to an embodiment, an electronic device (e.g., the electronic device 101 of FIG. 1, the electronic device 200 of FIGS. 2A and 2B, the electronic device 300 of FIG. 3, or the first electronic device 1710 or the second electronic device 1720 of FIG. 17) may include a blood glucose sensor (e.g., the blood glucose sensor of the sensor module 176 of FIG. 1) configured to generate a blood glucose value by measuring a blood glucose level of a user, and at least one processor (e.g., the processor 120 of FIG. 1 or the processor of FIGS. 2A and 2B) configured to control the electronic device, wherein the processor may generate logs for the blood glucose value of the user obtained through the blood glucose sensor (e.g., operation 420 of FIG. 4), determine a target event based on a target blood glucose value of the user obtained through the blood glucose sensor and the logs (e.g., operation 430 of FIG. 4), and output a message suggesting a target behavior for the target event (e.g., operation 450 of FIG. 4).

According to an embodiment, the processor may obtain a first blood glucose value at a first time through the blood glucose sensor (e.g., operation 610 of FIG. 6), obtain a second blood glucose value at a second time through the blood glucose sensor (e.g., operation 620 of FIG. 6), determine a type of a behavior performed by the user based on the first blood glucose value and the second blood glucose value (e.g., operation 630 of FIG. 6), and generate a log for the behavior based on the first blood glucose value, the second blood glucose value, and the type of the behavior (e.g., operation 640 of FIG. 6).

According to an embodiment, the electronic device may further include an acceleration sensor (e.g., the acceleration sensor of the sensor module 176 of FIG. 1) configured to measure acceleration information about a motion of the electronic device, wherein the processor may determine the type of the behavior based on the acceleration information.

According to an embodiment, the electronic device may further include a body temperature sensor configured to generate body temperature information by measuring a body temperature of the user, wherein the processor may determine the type of the behavior based on the body temperature information.

According to an embodiment, the processor may determine the type of the behavior to be having a meal when the first blood glucose value is above a preset average blood glucose range.

According to an embodiment, the processor may adjust an operating interval of the blood glucose sensor when the first blood glucose value is above the preset average blood glucose range.

According to an embodiment, the processor may calculate a fasting blood glucose value of the user (e.g., operation 810 of FIG. 8), and set the average blood glucose range based on the calculated fasting blood glucose value (e.g., operation 820 of FIG. 8).

According to an embodiment, the log for the type of the behavior may further include at least one of a blood pressure and a heart rate of the user.

According to an embodiment, the electronic device may further include an acceleration sensor configured to measure acceleration information about a motion of the electronic device, wherein the processor may obtain first acceleration information at a third time through the acceleration sensor (e.g., operation 920 of FIG. 9), obtain second acceleration information at a fourth time through the acceleration sensor (e.g., operation 930 of FIG. 9), determine a type of a behavior performed by the user based on the first acceleration information and the second acceleration information (e.g., operation 940 of FIG. 9), and generate a log for the behavior based on biometric information of the user obtained while the behavior is performed (e.g., operation 950 of FIG. 9).

According to an embodiment, the biometric information may include at least one of a blood glucose, a heart rate, a body temperature, a blood pressure, a stress level, and a consumed energy of the user.

According to an embodiment, the processor may determine whether the target blood glucose value is greater than or equal to a highest blood glucose value predetermined based on the logs (e.g., operation 1110 of FIG. 11), and determine the target event to be having an abnormal meal when the target blood glucose value is greater than or equal to the highest blood glucose value (e.g., operation 1120 of FIG. 11).

According to an embodiment, the processor may determine a blood glucose reduction time based on the target blood glucose value (e.g., operation 1210 of FIG. 12), and determine the target event to be having an abnormal meal (e.g., operation 1230 of FIG. 12) when the determined blood glucose reduction time is greater than or equal to a preset reference blood glucose reduction time.

According to an embodiment, the processor may determine the target event to be excessively exercising (e.g., operation 1320 of FIG. 13) when the target blood glucose value is less than or equal to a preset lowest blood glucose value.

According to an embodiment, the electronic device may be a wearable device.

According to an embodiment, a method, performed by an electronic device (e.g., the electronic device 101 of FIG. 1, the electronic device 200 of FIGS. 2A and 2B, the electronic device 300 of FIG. 3, or the first electronic device 1710 or the second electronic device 1720 of FIG. 17), may include generating logs for a blood glucose value of a user obtained through a blood glucose sensor (e.g., the blood glucose sensor of the sensor module 176 of FIG. 1) of the electronic device (e.g., operation 420 of FIG. 4), determining a target event based on a target blood glucose value of the user obtained through the blood glucose sensor and the logs (e.g., operation 430 of FIG. 4), and outputting a message suggesting a target behavior for the target event (e.g., operation 450 of FIG. 4).

According to an embodiment, an electronic device (e.g., the electronic device 101 of FIG. 1, the electronic device 200 of FIGS. 2A and 2B, the electronic device 300 of FIG. 3, or the first electronic device 1710 or the second electronic device 1720 of FIG. 17) may include a communication module (e.g., the communication module 190 of FIG. 1, comprising communication circuitry) configured to exchange data with an external device, and at least one processor (e.g., the processor 120 of FIG. 1) configured to control the electronic device,
wherein the processor may receive information about a blood glucose value of a user from an additional device (e.g., the electronic device 102 of FIG. 1, the electronic device 200 of FIGS. 2A and 2B, the electronic device 300 of FIG. 3, or the second electronic device 1720 of FIG. 17) through the communication module, generate logs for the blood glucose value of the user based on information about the blood glucose value, determine a target event based on a target blood glucose value of the user and the logs, and output a message suggesting a target behavior for the target event.

“Based on” as used herein covers based at least on.

According to an embodiment, the processor may output the message by transmitting the message to the additional device through the communication module, and the message may be output by the additional device.

According to an embodiment, the processor may determine a type of a behavior performed by the user at a time when the blood glucose value is obtained, and generate a log for the blood glucose value based on the blood glucose value and the type of the behavior.

According to an embodiment, the processor may determine the type of the behavior performed by the user based on acceleration information obtained by an acceleration sensor and body temperature information obtained by a body temperature sensor.

The embodiments described herein may be implemented using a hardware component, a software component and/or a combination thereof. A processing device may be implemented using one or more general-purpose or special-purpose computers, such as, for example, a processor, a controller and an arithmetic logic unit (ALU), a DSP, a microcomputer, a field-programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may be configured to act as one or more software modules in order to perform the operations of the above-described examples, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents. While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

Accordingly, other implementations are within the scope of the following claims.

1. An electronic device comprising:
a blood glucose sensor configured to generate a blood glucose value at least by measuring a blood glucose level of a user;
at least one processor; and
memory storing instructions that, when executed by the at least one processor individually and/or collectively, cause the electronic device to:
generate logs for the blood glucose value of the user obtained via the blood glucose sensor,
determine a target event based on a target blood glucose value of the user obtained through the blood glucose sensor and the logs, and
output a message suggesting a target behavior for the target event.

2. The electronic device of claim 1, wherein
the instructions, when executed by the at least one processor individually or collectively, cause the electronic device to:
obtain a first blood glucose value at a first time through the blood glucose sensor,
obtain a second blood glucose value at a second time through the blood glucose sensor,
determine a type of a behavior performed by the user based on the first blood glucose value and the second blood glucose value, and
generate a log for the behavior based on the first blood glucose value, the second blood glucose value, and the type of the behavior.

3. The electronic device of claim 2, further comprising:
an acceleration sensor configured to measure acceleration information about a motion of the electronic device,
wherein the instructions, when executed by the at least one processor individually or collectively, cause the electronic device to:
determine the type of the behavior based on the acceleration information.

4. The electronic device of claim 2, further comprising:
a body temperature sensor configured to generate body temperature information by measuring a body temperature of the user,
wherein the instructions, when executed by the at least one processor individually or collectively, cause the electronic device to determine the type of the behavior based on the body temperature information.

5. The electronic device of claim 2, wherein
the instructions, when executed by the at least one processor individually or collectively, cause the electronic device to:
determine the type of the behavior to be having a meal when the first blood glucose value is above a preset average blood glucose range.

6. The electronic device of claim 5, wherein
the instructions, when executed by the at least one processor individually or collectively, cause the electronic device to:
adjust an operating interval of the blood glucose sensor when the first blood glucose value is above the preset average blood glucose range.

7. The electronic device of claim 5, wherein
the instructions, when executed by the at least one processor individually or collectively, cause the electronic device to:
calculate a fasting blood glucose value of the user, and
set the average blood glucose range based on the calculated fasting blood glucose value.

8. The electronic device of claim 2, wherein
the log for the type of the behavior further comprises at least one of a blood pressure and a heart rate of the user.

9. The electronic device of claim 1, further comprising:
an acceleration sensor configured to measure acceleration information about a motion of the electronic device,
the instructions, when executed by the at least one processor individually or collectively, cause the electronic device to:
obtain first acceleration information at a third time through the acceleration sensor,
obtain second acceleration information at a fourth time through the acceleration sensor,
determine a type of a behavior performed by the user based on the first acceleration information and the second acceleration information, and
generate a log for the behavior based on biometric information of the user obtained while the behavior is performed.

10. The electronic device of claim 9, wherein
the biometric information comprises at least one of a blood glucose, a heart rate, a body temperature, a blood pressure, a stress level, and a consumed energy of the user.

11. The electronic device of claim 1, wherein
the instructions, when executed by the at least one processor individually or collectively, cause the electronic device to:
determine whether the target blood glucose value is greater than or equal to a highest blood glucose value predetermined based on the logs, and
determine the target event to be having an abnormal meal when the target blood glucose value is greater than or equal to the highest blood glucose value.

12. The electronic device of claim 1, wherein
the instructions, when executed by the at least one processor individually or collectively, cause the electronic device to:
determine a blood glucose reduction time based on the target blood glucose value, and
determine the target event to be having an abnormal meal when the determined blood glucose reduction time is greater than or equal to a preset reference blood glucose reduction time.

13. The electronic device of claim 1, wherein
the instructions, when executed by the at least one processor individually or collectively, cause the electronic device to:
determine the target event to be excessively exercising when the target blood glucose value is less than or equal to a preset lowest blood glucose value.

14. The electronic device of claim 1, wherein
the electronic device is a wearable device.

15. A method, performed by an electronic device, the method comprising:
generating logs for a blood glucose value of a user obtained through a blood glucose sensor of the electronic device;
determining a target event based on a target blood glucose value of the user obtained through the blood glucose sensor and the logs; and
outputting a message suggesting a target behavior for the target event.

16. A computer-readable storage medium storing a program to perform the method of claim 15.

17. An electronic device comprising:
a communication module, comprising communication circuitry, configured to exchange data with an external device; and
at least one processor; and
memory storing instructions that, when executed by the at least one processor individually and/or collectively, cause the electronic device to:
receive information about a blood glucose value of a user from an additional device through the communication module,
generate logs for the blood glucose value of the user based on information about the blood glucose value,
determine a target event based on a target blood glucose value of the user and the logs, and
output a message suggesting a target behavior for the target event.

18. The electronic device of claim 17, wherein
the instructions, when executed by the at least one processor individually or collectively, cause the electronic device to output the message by controlling for transmitting the message to the additional device through the communication module, and
the message is output by the additional device.

19. The electronic device of claim 17, wherein
the instructions, when executed by the at least one processor individually or collectively, cause the electronic device to:
determine a type of a behavior performed by the user at a time when the blood glucose value is obtained, and
generate a log for the blood glucose value based on the blood glucose value and the type of the behavior.

20. The electronic device of claim 19, wherein
the instructions, when executed by the at least one processor individually or collectively, cause the electronic device to determine the type of the behavior performed by the user based on acceleration information obtained by an acceleration sensor and body temperature information obtained by a body temperature sensor.
